(19) United States
(12) Patent Application Publication
(10) Pub. No.: US 2013/0274763 A1
(43) Pub. Date: Oct. 17, 2013

(54) METHODS AND DEVICES FOR FACET JOINT PREPARATION AND IMPLANT DELIVERY

(71) Applicants: Susan J. Drapeau, Collierville, TN (US); Daniel A. Shimko, Memphis, TN (US)

(72) Inventors: Susan J. Drapeau, Collierville, TN (US); Daniel A. Shimko, Memphis, TN (US)

(73) Assignee: Warsaw Orthopedic, Inc., Warsaw, IN (US)

(21) Appl. No.: 13/446,156

(22) Filed: Apr. 13, 2012

(51) Int. Cl. A61B 17/50

(52) U.S. Cl. 606/132

(57) ABSTRACT

A system for treating a tissue site is provided, the system including a graft preparation device for treating a tissue site, the preparation device including an elongate inner member having a proximal end connected to a handle and a distal end including an abrasive end configured for abrading the tissue site, and an elongate outer member movably attached to the elongate inner member, the elongate outer member including a tapered end. The elongate outer member is movable between a first position for covering the abrasive end and a second position for exposing the abrasive end. Following treatment of the tissue site using the graft preparation device to remove soft tissue and cause bleeding, the tissue site is distracted and an implant body is inserted into the tissue site.

### BACKGROUND

The facet joint is a synovial joint between the superior articular process of one vertebra and the inferior articular process of the vertebra directly above it. There are two facet joints in each spinal motion segment. The biomechanical function of each pair of facet joints is to guide and limit movement of the spinal motion segment. In the lumbar spine, for example, the facet joints function to protect the motion segment from anterior shear forces, excessive rotation and flexion. These functions can be disrupted by degeneration, dislocation, fracture, injury, instability from trauma, osteoarthritis, and surgery. In the thoracic spine the facet joints function to restrain the amount of flexion and anterior translation of the corresponding vertebral segment and function to facilitate rotation.

In large part due to the mechanical nature of their function, all joints undergo degenerative changes with the wear and tear of age. This is particularly true for joints in the spine, and the facet joint in particular. Chronic back problems cause pain and disability for a large segment of the population and adverse spinal conditions are characteristic of advancing age. With aging, generally comes an increase in spinal stenosis (including, but not limited to, central canal and lateral stenosis), and facet arthropathy. Spinal stenosis results in a reduction of foraminal area (i.e. the available space for the passage of nerves and blood vessels), which compresses the cervical nerve roots and causes radicular pain. Extension and ipsilateral rotation of the neck further reduces the foraminal area and contributes to pain, nerve root compression, and neural injury.

Neck and arm pain is a common ailment of the aging spine due to disc herniations, facet arthropathy and thickening of spinal ligaments which narrow spinal canal dimensions. This results in compression of the spinal cord or nerve roots, or both. Radicular pain is typically due to disc herniation and foraminal narrowing, which compresses the cervical nerve roots and causes radicular pain. Extension and ipsilateral rotation of the neck further reduces the foraminal area and contributes to pain, nerve root compression, and neural injury. Neck flexion generally increases the foraminal area.

Cervical disc herniations predominantly present upper extremity radicular symptoms. The vast majority of these herniations do not have an associated neurological deficit and present pain only. A well-described treatment for cervical disc herniations is closed traction. There are a number of marketed devices that alleviate pain by pulling on the head to increase foraminal height.

Cervical disc herniations have been treated with anterior and posterior surgery. The vast majority of these surgeries are performed through an anterior approach, which requires a spinal fusion. These surgeries are expensive and beget additional surgeries due to change in biomechanics of the neck. There is a three percent incidence of re-operation after cervical spine surgery.

Spine fusion surgery is migrating to a more mid-line minimal access approach. Solid fusion in the facets can help to stabilize a motion segment and potentially augment instrumentation. However, simple placement of a graft into the facet space may not lead to successful fusion.

There is a need for minimally invasive methods and devices for accessing, preparing and distracting the facet joint to increase foraminal height and improve fusion of an implant with the facet joint, thus ultimately reducing radicular symptoms for patients with soft and hard disc disease.

### SUMMARY

Methods and devices for preparation and distraction of a graft site, as well as delivery of an implant to the graft site are provided. In some embodiments, devices are provided for preparing a cervical graft site, enlarging a facet joint and delivering a graft implant to the facet joint. In some embodiments, methods and devices are provided for preparing a facet joint of a spine for receiving a bone graft material, the method and devices comprise removing cartilage tissue from at least a portion of the facet joint and disrupting the surface of the portion of the facet so as to cause the surface of the facet to bleed; and then inserting the bone graft material into a bone graft tissue site at or near the facet joint. By disrupting the facet joint and causing it to bleed, the bone graft material can integrate better into the facet joint.

In one embodiment, a graft preparation device for treating a tissue site is provided, the device comprising an elongate inner member having a proximal end connected to a handle and a distal end including an abrasive end configured for abrading the tissue site; and an elongate outer member movably attached to the elongate inner member, the elongate outer member including a tapered end, wherein the elongate outer member is movable between a first position for covering the abrasive end and a second position for exposing the abrasive end.

In another embodiment, a method for treating a tissue site is provided, the method comprising the steps of inserting a tissue site preparation device into a tissue site; abrading the tissue site to remove soft tissue and cause bleeding at the tissue site; distracting the tissue site and inserting an implant body at the tissue site.

In yet another embodiment, a system for treating a tissue site is provided, the system comprising a graft preparation device for treating a tissue site, the device comprising an elongate inner member having a proximal end connected to a handle and a distal end including an abrasive end configured for abrading the tissue site; and an elongate outer member movably attached to the elongate inner member, the elongate outer member including a tapered end, wherein the elongate outer member is movable between a first position for covering the abrasive end and a second position for exposing the abrasive end; a distraction device including a sleeve member movable attached to a shank and jointed distraction members connected to a distal end of the sleeve member, wherein the jointed distraction members include contact elements and wherein the distraction device is configured to distract the tissue site following preparation of the tissue site by the graft preparation device; and an implant delivery device including a sleeve member movably affixed to a shank, wherein a distal end of the shank includes two arms separated by a groove, wherein the implant delivery device is configured to insert an implant body into the tissue site following preparation and distraction of the tissue site.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a side view of an exemplary facet preparation device according to an aspect of the present application;

FIG. 2 illustrates the facet preparation device of FIG. 1 in a retracted position according to an aspect of the present application;

FIG. 3 illustrates a side view of an exemplary shank tip according to an alternative embodiment;

FIG. 4 is a cross-sectional view taken along lines 4-4 of FIG. 3;

FIG. 5 illustrates a side view of an exemplary shank tip according to another alternative embodiment;

FIG. 6 is a cross-sectional view taken along lines 6-6 of FIG. 5;

FIG. 7 illustrates a side view of an exemplary distraction device according to an aspect of the present application;

FIG. 8 illustrates a side view of a distraction mechanism according to an alternative embodiment;

FIG. 9 illustrates a side view of an implant delivery device in a grasping position according to an aspect of the present application;

FIG. 10 illustrates the implant delivery device of FIG. 9 in a releasing position according to an aspect of the present application; and

FIG. 11 illustrates an exemplary graft implant.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a facet engaging feature” includes one, two, three or more facet engaging feature(s).

Reference will now be made in detail to certain embodiments of the application, examples of which are illustrated in the accompanying drawings. While the application will be described in conjunction with the illustrated embodiments, it will be understood that they are not intended to limit the application to those embodiments. On the contrary, the application is intended to cover all alternatives, modifications, and equivalents, which may be included within the application as defined by the appended claims.

The headings below are not meant to limit the disclosure in any way; embodiments under any one heading may be used in conjunction with embodiments under any other heading.

### Devices and Methods

Each spinal column vertebrae has two facet joints, with facet joints between adjacent facets of the vertebrae. The spinal cord passes vertically through the aligned vertebrae with peripheral nerves passing from the spinal cord outwardly through the spinal column through foraminal openings to predestined locations in the human body. When facet joints become narrowed, usually from disc degeneration, the foraminal openings are reduced in size pinching the nerve and causing pain to the individual.

Methods and devices are provided in the present application for preparing an intra-facet space for a graft implant via a minimal access approach without disruption of surrounding tissue, as well as distraction of the facet space and subsequent delivery of the implant to the prepared site. In various embodiments, a graft preparation instrument, a distraction device and a graft delivery device are provided. According to some embodiments, a graft preparation device is provided configured for preparing or treating the area of the target tissue site (e.g., a facet joint), by abrading, disrupting and/or roughening at least a portion of the area of the target tissue site, e.g., to remove cartilage and other soft tissue from at least a portion of the site and causing the surface of the tissue site to bleed. Advantageously, such treatment and preparation of the intra-facet site accordingly allows improved integration of a subsequently inserted bone graft material within the facet joint.

All components of the devices shown herein and any embodiments contemplated for the same may comprise metallic materials, polymer materials, or combinations thereof, and may be adapted to be reusable and/or disposable. In some embodiments, a length of the graft preparation devices, distraction devices and graft delivery devices of the present application may comprise, e.g., about 10 inches to about 18 inches and have a diameter of about 8 mm to about 15 mm, or about 11 mm to about 12 mm.

Each of the devices in the present application may be used in conjunction with an access system such as an access port (e.g., a minimal access port), for example, dilators which may progressively increase in diameter size. For example, dilators may be inserted sequentially (smaller to larger) through the tissue site to gradually separate, or split, and open the tissue to create an opening large enough for the surgical devices to be used. The dilator tubes may be used to maintain the opening while surgical devices to reach the site are inserted within the tubes. A kit comprising a graft preparation instrument, a distraction device and a graft delivery device may be provided.

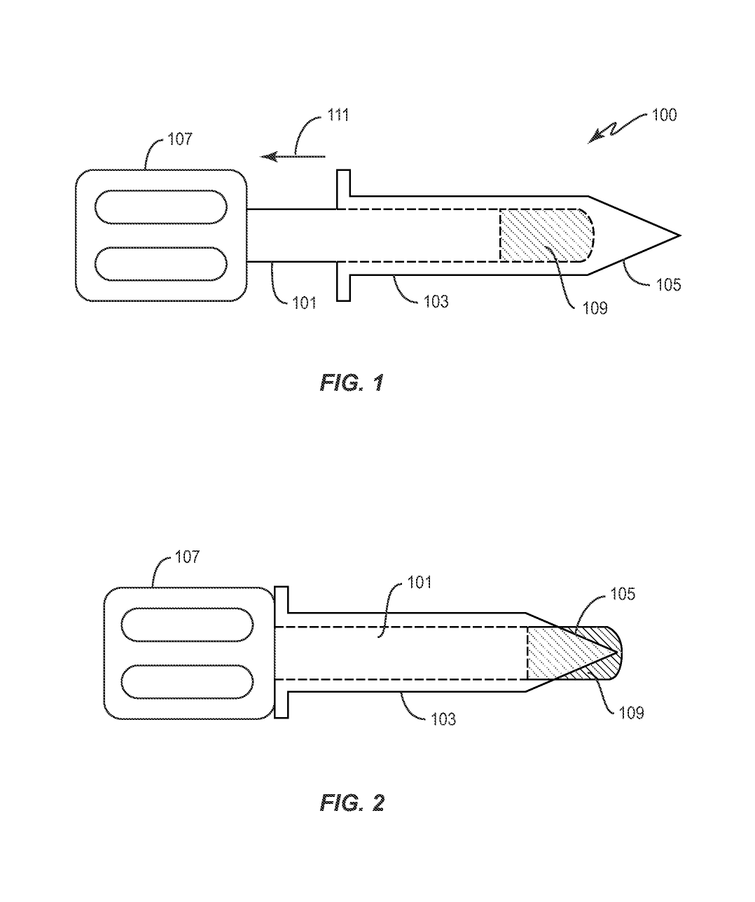

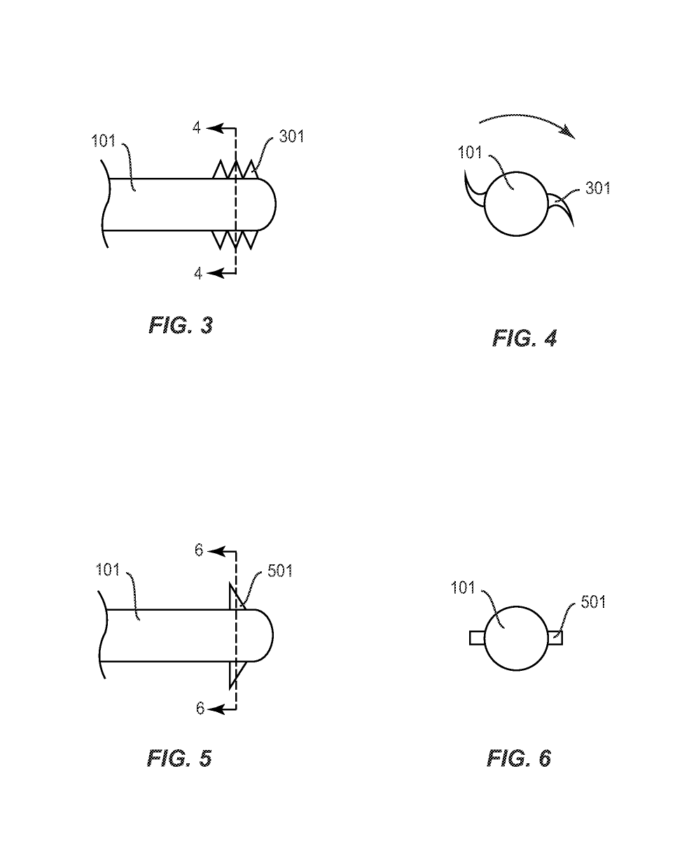

Referring to the figures, FIGS. 1-2 show an exemplary graft preparation device according to an aspect of the present application, while FIGS. 3-6 provide views of exemplary abrasive ends of the preparation device according to various alternative embodiments.

Pursuant to one aspect of the present application, a graft/tissue site preparation device 100 is provided having an elongate inner member 101 having a proximal end affixed to a handle 107. The handle 107 may comprise any shape and material and is configured to improve a user's grip to facilitate effective and precise rotation and manipulation of the device 100 by a user.

A distal end 109 of the elongate inner member 101 may comprise an abrasive end having facet disruption features, such as, for example, a bur-type tip 109, as shown in FIGS. 1 and 2. The elongate inner member 101 may be comprises any rigid material, such as stainless steel and is preferably substantially cylindrical in shape with a circular cross-section. In some embodiments, the bur type tip may extend from about 5 mm to about 3 cm along the distal end of the elongate inner member 101, but any desired length of the bur tip 109 may be contemplated. The bur-type tip may be formed by e.g., diamond bur particles bonded by means of electroplating to the elongate inner member 101. Alternative compositions and configurations of the bur-type tip may be contemplated to achieve a desired abrasive effect on a tissue site.

An elongate outer member 103 may be provided movable engaged to the outer surface of the elongate inner member 101. The elongate outer member 103 may be of various shapes including, but not limited to, cylindrical or round. In some embodiments, the elongate outer member 103 may comprise a hollow cylindrical tube having a diameter just slightly larger than the diameter of the elongate inner member 101 so as to be snugly fitted thereon. The elongate outer member 103 may include a tapered end 105 to facilitate insertion of the device 100 and distraction of the target joint site. The tapered end 105 may include an aperture and/or comprises deformable material to permit the distal end 109 to emerge from the elongate outer member 103 when the elongate outer member 103 is in a retracted position, as shown in FIG. 2 and described further below. In some embodiments, the length of the elongate outer member 103 is sufficient to at least cover the bur-type tip of distal end 109 when in an extended position, as shown in FIG. 1, but enables the bur tip of distal end 109 to be exposed when the elongate outer member 103 is in a retracted position as shown in FIG. 2.

In some embodiments, the preparation device 100 may be formed of sufficiently small diameter to be inserted through an access system such as an access port/minimal access port, for example, specially designed metal tubes such as ‘working cannulas’ or dilators, which may progressively increase in diameter size. For example, dilators may be inserted sequentially (smaller to larger) through the tissue site to gradually separate, or split, and open the tissue to create an opening large enough for surgical tools to be used. The dilator tubes may be used to maintain the opening while surgical tools to reach the site are inserted within the tubes.

In one embodiment, the elongate outer member 103 may be extended away from the handle 107 to a first position as shown in FIG. 1 to cover the abrasive end 109 and to cause the tapered end 105 to protrude. This enables the device 100 to be easily inserted into a tissue site desired to be prepared for receipt of the bone graft material, such as an intra-facet space comprising a facet capsule between superior and inferior facet faces. Once the preparation device 100 is inserted into a tissue site, the elongate outer member 103 may then be retracted towards the handle 107 to a second position as shown in FIG. 2 to cause the distal end 109 to protrude from the tapered end 105. In some embodiments, this exposes the abrasive surface of the distal end 109 (e.g., the bur tip) at the tissue site.

The surgeon may then apply energy to the handle 107 of the device 100 to cause the exposed abrasive end 109 to rub the surfaces of the tissue site, thus abrading the tissue surfaces. In some embodiments, the abrasive end 109 is rotated to engage soft tissue such as cartilage around the facet surfaces, and/or the abrasive end 109 is withdrawn and/or inserted repeatedly to allow it to scrape the tissue site and cause the surface of the facet to be disrupted and/or roughened and preferably, cause it to bleed.

Alternative configurations of the abrasive end 109 (of FIG. 1 and FIG. 2) may be contemplated. For example, as shown in FIGS. 3 and 5, the abrasive end 109 may include protrusions such as toothed spikes 301 or angled spikes 501. These protrusions may be on elongate inner member 101. The protrusions may be formed to oppose each other, e.g., to protrude from the distal end 109 on at least its top and bottom surfaces. Exemplary cross-sectional views of the toothed spikes 301 and angled spikes 501 are shown in FIGS. 4 and 6, respectively. These spikes may be on elongate inner member 101. For example, toothed spikes 301 may be curved to cause the abrasive end 109 to bore into the tissue site during rotation of the device 100 in a first direction (e.g., a clockwise direction). Subsequent rotation of the device 100 in an opposite direction (e.g., counterclockwise direction) may cause it to be removed or withdrawn from the tissue site. In other embodiments, angled spikes 501 may cause effective scraping of the tissue site upon withdrawal of the device 100 from the tissue site. Alternative embodiments of the abrasive end 109 such as e.g., a cruciate extension forced from the interior of the elongate inner member 101 or any other abrasive means formed on the distal end 109 may be contemplated. For example, the distal end 109 may comprise oval, oblong, triangular, rectangular, square, polygonal, irregular, tubular, non-tubular, uniform, non-uniform, variable and/or tapered protrusions that assist in removing tissue and/or scrapping bone.

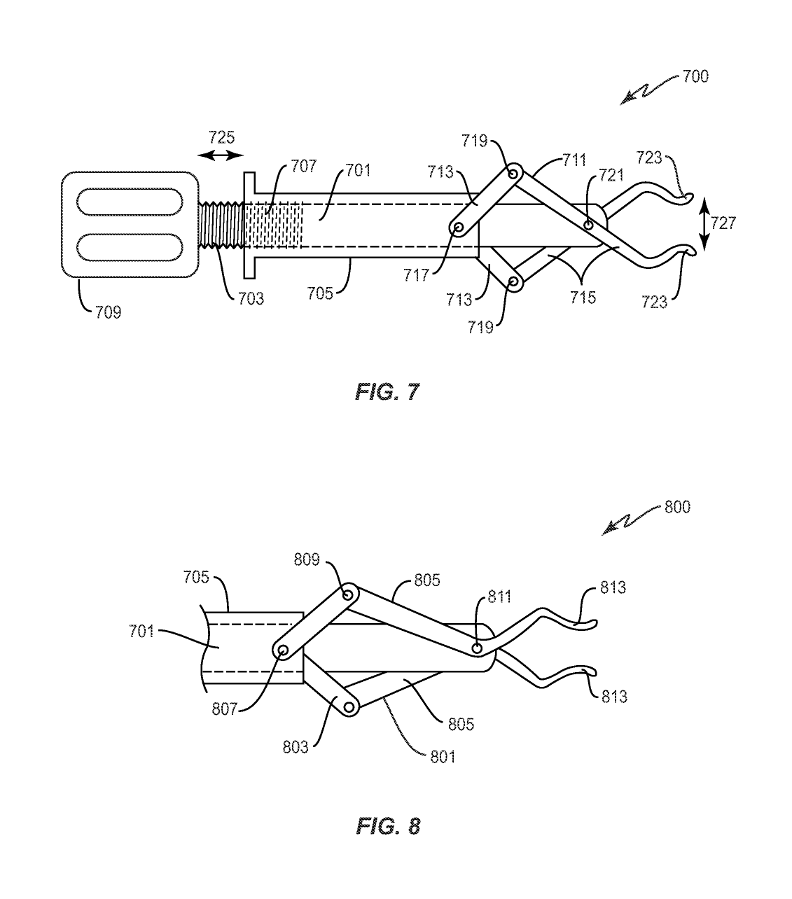

Following preparation of the tissue site, distraction of the facet joint may be performed using a distraction device 700, e.g., as shown in FIG. 7. In some embodiments, the distraction device 700 comprises a shank 701 connected to a handle 709 and having a threaded portion 703. A sleeve member 705 may be provided movably affixed to the shank 701. The sleeve member 705 may be slidable and/or rotatable with respect to the shank 701. In some embodiments, the sleeve member may have a corresponding threaded interior portion 707 configured to threadably engage the threaded portion 703 of the shank 701. Accordingly, in some embodiments, the sleeve 705 is enabled to be incrementally extended and retracted 725 along the shank 701 via the mated threaded portions 703, 707.

A distal end of the sleeve 705 may include a first joint 717 for connection of the sleeve 705 with jointed distraction members 711. The jointed distraction members 711 may be comprises a pair of first arms 713, each first arm 713 being pivotally connected to each of a pair of second arms 715, with each of the connected first and second arms 713, 715 being situated on opposing sides of the sleeve 705. The pivotal connection between the first and second arms 713, 715 may be provided via a second joint 719. A third joint 721 may be provided to pivotally connect each of the second arms 711 to a distal end of the shank 701.

A distal end of each of the second arms 711 may be shaped to form contact elements 723, which are configured to be movable in direction 727 to contact and apply pressure within the facet joint to provide effective separation and distraction of the facet joint site. The separation can occur in both the vertical and horizontal planes of the facet joint resulting in vertical distraction and forward/anterior translation of the superior vertebrae relative to the inferior vertebrae. Advantageously, the facet joint distraction and forward translation can cause an increase in foraminal area and thus reduce nerve root compression and associated symptoms. The contact elements 723 may be curved as shown in FIGS. 7 and 8 or comprise any other shape or configuration.

In some embodiments as shown in FIG. 7, each second arm 715 may be formed in a general V-shape, namely to be substantially straight along its length from the second joint 719 to past its connection with joint 721, and thereafter curving inwards to form the contact elements 723. Accordingly, in use, the sleeve 705 may be moved in a first direction (e.g., towards the handle 709), which causes straightening of the first arm 713 relative to the second arm 715, thus causing the contact elements 723 to contract, e.g., be moved closer together. This can allow insertion or withdrawal of the device 700 from the tissue site. Conversely, the sleeve 705 may be movable in a second direction (e.g., away from the handle 709) which will cause the increased angulation of the first arm 713 relative to the second arm 715, thus causing the contact elements 723 to expand, i.e., move away from each other in direction 727. This can provide effective distraction of the facet joint and space for bone graft material to be placed therein.

FIG. 8 depicts an alternative embodiment of a distraction device 800 showing jointed distraction members 801 according to an alternative configuration. In this embodiment, the device 800 is configured to operate in a reverse manner to the device shown in FIG. 7. The distraction members 801 may comprise a pair of first arms 803, each first arm 803 being pivotally connected to each of a pair of second arms 805. A first joint 807 may be provided to enable pivotal connection of the first arms 803 relative to the sleeve 705. A second joint 809 may be provided to pivotally connect each of the first arms 803 with each of the second arms 805. A third joint 811 may be provided to pivotally connect the second arms 805 to the shank 701.

In the exemplary embodiment of FIG. 8, each of the second arms 805 may be formed in a ‘zig-zag’ shape, namely, to be substantially straight only from the second joint 809 to the third joint 811. After the third joint, each second arm 805 may be formed to curve or bend outwards away from each other, and then bend inwards towards each other to form the contact elements 813. Accordingly, in use, the sleeve 705 may be rotated in a first direction (e.g., towards the handle 709 of FIG. 7), which causes straightening of the first arm 803 relative to the second arm 805, thus causing the contact elements 813 to be moved apart from each other. This can enable effective distraction of the facet joint. Rotating the sleeve 705 in a second direction (e.g., away from the handle 709) will cause the increased angulation of the first arm 803 relative to the second arm 805, thus causing the contact elements 813 to move closer together and will allow the disc to retract. Slidable and/or rotable shank is shown as 701. This embodiment of the device can allow insertion or withdrawal of the device 800 through, e.g., a working cannula or other access system.

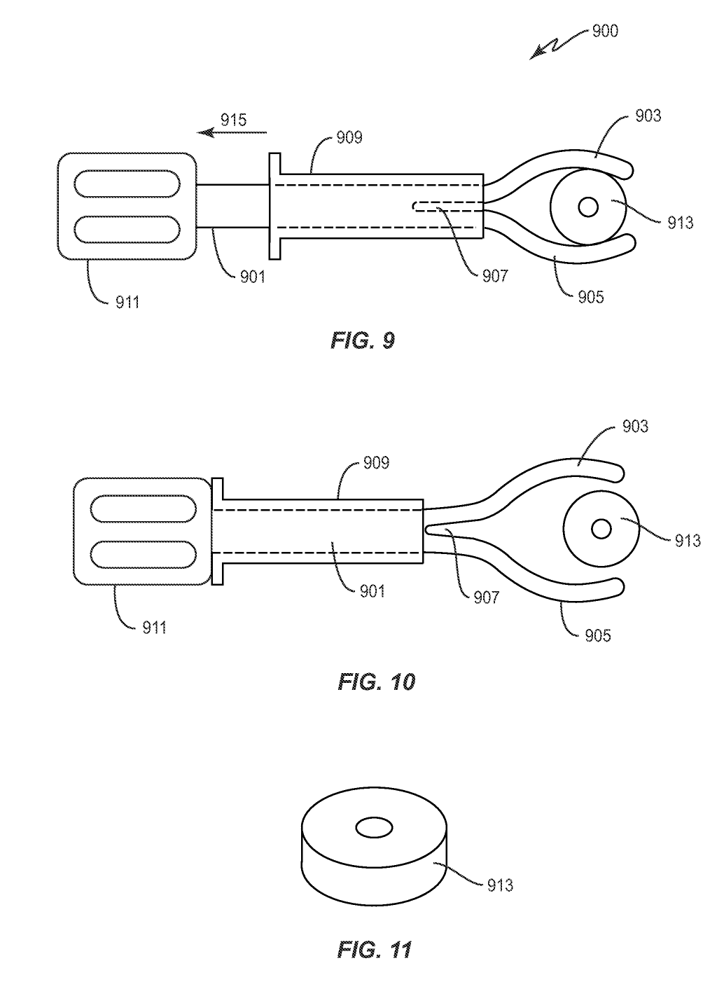

Following preparation and distraction of a tissue site, in some embodiments, insertion of an implant may be performed using an implant delivery device. FIG. 9 illustrates a side view of an exemplary implant delivery device 900 in a grasping position according to an aspect of the present application. The delivery device 900 comprises a shank 901 movably affixed to a handle 911 at a proximal end. A distal end of the shank 901 may comprise two curved arms 903, 905 separated by a groove 907. The arms 903, 905 may comprise resilient material, such as nitinol, polymer, steel, titanium, carbon, or any other rigid yet elastic, bendable material, which has shape memory properties to enable it to spring back to its original form.

A sleeve member 909 may be provided slidably affixed to the exterior of the shank 901. In a first position as shown in FIG. 9, the sleeve 909 may be extended to abut the curved arms 903, 905, thus compressing the groove 907 to a substantially closed position. This causes the arms 903, 905 to be pushed closer together, thus enabling them to be used to grasp objects, such as a graft implant body 913, for delivery to a tissue site. The sleeve 909 is movable in a direction 915 toward the handle 911, which spaces apart arms 903 and 905 to release the graft implant body 913.

FIG. 10 illustrates the implant delivery device 900 of FIG. 9 in a second position (‘releasing’ position) according to an aspect of the present application. As shown, the sleeve 909 may be retracted towards the handle 911, thus releasing the compression of the groove 907. This causes the arms 903, 905 to be released and expand away from each other, thus releasing any object, which is grasped between them. Accordingly, a graft implant body 913 may be deposited at the tissue site. Shank 901 is shown within sleeve 909.

While an exemplary implant body 913 has been depicted in the shape of a flattened cylinder herein, permanent implants can vary in geometry, material, and fixation mechanism. For example with respect to geometry, a wedge shaped implant can provide for a greater height of the posterior aspect of the implant relative to the anterior aspect of the implant. The wedge can also provide for uniform dimensions at the lateral and medial aspects of the implant. The wedge shape may result in a translating vector force and a separating vector force that results in both subluxation and distraction, thereby increasing the foraminal space more fully. A double wedged implant can provide greater height of the posterior aspect of the implant relative to the anterior aspect of the implant in addition to greater height of the lateral aspect of the implant relative to the medial aspect of the implant. Other geometrical variations can include a flat rectangular shape, an oval pill shape, a concave superior surface, a concave inferior surface, a convex superior surface, a convex inferior surface, a convex anterior surface, a concave anterior surface, a convex posterior surface, and a concave posterior surface.

With respect to graft materials, several materials can be provided including steel, PEEK, carbon, allograft, polymer, and silicone. The graft material may include various fixation mechanisms or friction structures, such as a plurality of threads, protrusions, ridges and the like. For example, aggressive teeth can be provided with a directional orientation positioned to achieve optimal fixation relative to the natural biomechanics of various sections of the spine. The teeth can be long enough to gain purchase in the cortical bone of the facet surfaces. Cleats can also be provided that have a less aggressive profile than the shark teeth but still allow for directional orientation for the same reasons described above. These cleats can also be capable of anchoring in the hard cortical bone of the facet surface. Additionally, a roughened pore surface can be provided to prevent free sliding of the implant within the facet joint. These surfaces can be roughened and coated with commercially available resurface chemicals that would create friction and prevent device migration.

According to some embodiments, the bone graft material may include various osteoinductive and/or osteogenic materials with or within the bone graft material. Such osteoinductive materials may be introduced before, during, or after insertion of the exemplary bone graft material, and may include (but are not necessarily limited to) autologous bone harvested from the patient receiving the implant, bone allograft, bone xenograft, any number of non-bone implants (for example ceramic, metallic, polymer), bone morphogenic protein, and/or bio-resorbable compositions. The osteogenic material may be selected from among many known to those skilled in the art. For example, the osteogenic material may comprise minerals such as calcium phosphate or calcium sulfate minerals, bone, including xenograft, allograft or autograft bone, or the like. The osteogenic material may also comprise demineralized bone matrix (DBM), osteoinductive factors such as bone morphogenetic proteins (for example human BMP-2 or human BMP-7 or heterodimers thereof) whether recombinantly produced or purified from tissues, LIM mineralization proteins (LMPs), or the like. The osteogenic material may also comprise a binder material such as blood, clottable blood fractions, platelet gel, collagen, gelatin, carboxymethyl cellulose, or other similar materials that will serve to bind together harder particles or materials such as mineral particles (for example bone or synthetic mineral particles).

One class of molecules suitable for one embodiment of the disclosure is growth factors. Growth factors suitable for use in the practice of the disclosure include but are not limited to bone morphogenic proteins, for example, BMP-2, rhBMP-2, BMP-4, rhBMP-4, BMP-6, rhBMP-6, BMP-7 (OP-1), rhBMP-7, GDF-5, and rhGDF-5. Bone morphogenic proteins have been shown to be excellent at growing bone and there are several products being tested. For example, rhBMP-2 delivered on an absorbable collagen sponge (INFUSE® Bone Graft, Medtronic Sofamor Danek, Memphis, Tenn.) has been used inside titanium fusion cages and resulted in successful fusion and can be used on a ceramic carrier to enhance bone growth in a posterolateral fusion procedure. rhBMP-2 can also be used on a carrier for acute, open fractures of the tibial shaft. BMP-7 (OP-1) also enhances bone growth in a posterolateral fusion procedure.

Additionally, suitable growth factors include, without limitation, LIM mineralization protein, platelet derived growth factor (PDGF), transforming growth factor β (TGF-β), insulin-related growth factor-I (IGF-I), insulin-related growth factor-II (IGF-II), fibroblast growth factor (FGF), and beta-2-microglobulin (BDGF II).

Any or all of the implants can be adapted as fusion type implants or motion preservation type devices. Implants with varying degrees of motion preservation can also be provided. In the case of a motion preservation type implant, the implant can have fixation mechanisms on one side to enable both temporary and permanent fixation to one surface of the facet joint while allowing the opposing facet surface to slide freely across the surface of the implant. The facet joint can be a naturally sliding joint and a distraction implant with fixation on only one side may accommodate the natural sliding of the facet. However, in some circumstances, a fusion type implant can be more suitable. In these circumstances the implant can include fixation mechanisms on both sides of the implant.

### Sterilization

The preparation device, distraction device and delivery device components may be lightweight, disposable and sterilizable. In various embodiments, one or more components of each device are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

In various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of each device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity.

Other methods may also be used to sterilize one or more components of each device, including, but not limited to, gas sterilization, such as, for example, with ethylene oxide or steam sterilization.

In various embodiments, a kit is provided for preparing and delivering an implant to a tissue site, the kit comprising: a sterilized graft preparation device, comprising an elongate inner member having a proximal end connected to a handle and a distal end including an abrasive end configured for abrading the tissue site; and an elongate outer member movably attached to the elongate inner member, the elongate outer member including a tapered end, wherein the elongate outer member is movable between a first position for covering the abrasive end and a second position for exposing the abrasive end; a sterilized distraction device including a sleeve member movable attached to a shank and jointed distraction members connected to a distal end of the sleeve member, wherein the jointed distraction members include contact elements and wherein the distraction device is configured to distract the tissue site following preparation of the tissue site by the graft preparation device; and a sterilized implant delivery device including a sleeve member movably affixed to a shank, wherein a distal end of the shank includes two arms separated by a groove, wherein the implant delivery device is configured to insert an implant body into the tissue site following preparation and distraction of the tissue site.

In various embodiments, a kit is provided which may include additional parts along with the preparation, distraction and delivery devices. The kit may include the preparation, distraction and delivery devices in a first compartment. The second compartment may include an access system, and any other instruments needed for the implant. A third compartment may include gloves, drapes, wound dressings and other procedural supplies for maintaining sterility of the implanting process, as well as an instruction booklet. A fourth compartment may include additional shanks having alternative shaped abrasive ends. Each tool may be separately packaged in a plastic pouch that is radiation sterilized. A cover of the kit may include illustrations of the implanting procedure and a clear plastic cover may be placed over the compartments to maintain sterility.

Although the present invention has been described with a certain degree of particularity, it is understood the disclosure has been made by way of example, and changes in detail or structure may be made without departing from the spirit of the invention as defined in the appended claims. It is to be understood that the features of any given embodiment can be combined with features of other embodiments and still be within the scope of the invention. Where functionality allows, interchanging certain features of one embodiment with another embodiment is within the scope of the present invention. Thus, it is intended that various embodiments cover other modifications and variations of various embodiments within the scope of the present teachings.

What is claimed is:

1. A graft preparation device for treating a tissue site, the device comprising: an elongate inner member having a proximal end connected to a handle and a distal end including an abrasive end configured for abrading the tissue site; and an elongate outer member movably attached to the elongate inner member, the elongate outer member including a tapered end, wherein the elongate outer member is movable between a first position for covering the abrasive end and a second position for exposing the abrasive end.

2. A graft preparation device of claim 1, wherein the abrasive end comprises a burr type tip.

3. A graft preparation device of claim 1, wherein the abrasive end includes protrusions formed to protrude from the abrasive end in at least opposing directions.

4. A graft preparation device of claim 3, wherein the protrusions are configured to enable the abrasive end to engage the tissue site during rotation of the device within the tissue site.

5. A graft preparation device of claim 3, wherein the protrusions are configured to scrape the tissue site during withdrawal of the device from the tissue site.

6. A graft preparation device of claim 1, wherein the device is configured to be inserted into an access port for accessing the tissue site.

7. A graft preparation device of claim 1, wherein the first position comprises the elongate outer member in an extended position relative to the elongate inner member and the second position comprises the elongate outer member in a retracted position relative to the elongate inner member.

8. A graft preparation device of claim 1, further comprising a distraction device including a sleeve member movably attached to a shank and jointed distraction members connected to a distal end of the sleeve member, wherein the jointed distraction members include contact elements and wherein the distraction device is configured to distract the tissue site following preparation of the tissue site by the graft preparation device.

9. A graft preparation device of claim 8, wherein the sleeve member is movable with respect to the shank in a first direction to cause the contact elements to contract and in a second direction to cause the contact elements to expand.

10. A graft preparation device of claim 8, further comprising an implant delivery device including a sleeve member movably affixed to a shank, wherein a distal end of the shank includes two arms separated by a groove, wherein the implant delivery device is configured to insert an implant body into the tissue site following preparation and distraction of the tissue site.

11. A graft preparation device of claim 10, wherein the sleeve member is movable with respect to the shank to a first position for compressing the two arms and to a second position to release the two arms.

12. A method for treating a tissue site, the method comprising the steps of: inserting a tissue site preparation device into a tissue site; abrading the tissue site to remove soft tissue and cause bleeding at the tissue site; distracting the tissue site and inserting an implant body at the tissue site.

13. A method for treating a tissue site of claim 12, wherein the tissue site comprises a facet joint.

14. A method for treating a tissue site of claim 12, wherein the tissue site preparation device includes an abrasive end configured for enabling abrasion of the tissue site at least during rotation or withdrawal of the preparation device.

15. A method for treating a tissue site of claim 12, wherein the step of inserting comprises inserting the preparation device through an access port for accessing the tissue site.

16. A method for treating a tissue site of claim 12, further comprising providing a distraction device for distracting the tissue site, the distraction device including a sleeve member movable attached to a shank and jointed distraction members connected to a distal end of the sleeve member, wherein the jointed distraction members include contact elements, wherein the sleeve member is movable with respect to the shank in a first direction to cause the contact elements to contract and in a second direction to cause the contact elements to expand.

17. A method for treating a tissue site of claim 12, further comprising providing an implant delivery device for inserting the implant body, the implant delivery device including a sleeve member movably affixed to a shank, wherein a distal end of the shank includes two arms separated by a groove, wherein the sleeve member is movable with respect to the shank to a first position for compressing the two arms and to a second position to release the two arms.

18. A system for treating a tissue site, the system comprising: (i) a graft preparation device for treating a tissue site, the device comprising an elongate inner member having a proximal end connected to a handle and a distal end including an abrasive end configured for abrading the tissue site; and an elongate outer member movably attached to the elongate inner member, the elongate outer member including a tapered end, wherein the elongate outer member is movable between a first position for covering the abrasive end and a second position for exposing the abrasive end; (ii) a distraction device including a sleeve member movable attached to a shank and jointed distraction members connected to a distal end of the sleeve member, wherein the jointed distraction members include contact elements and wherein the distraction device is configured to distract the tissue site following preparation of the tissue site by the graft preparation device; and/or (iii) an implant delivery device including a sleeve member movably affixed to a shank, wherein a distal end of the shank includes two arms separated by a groove, wherein the implant delivery device is configured to insert an implant body into the tissue site following preparation and distraction of the tissue site.

19. A system for treating a tissue site of claim 18, wherein each of the preparation device, distraction device and implant delivery device is configured to be inserted into an access port for accessing the tissue site.

20. A system for treating a tissue site of claim 18, wherein the abrasive end comprises at least one of a burr type tip or protrusions formed to protrude from the abrasive end.
